(19) United States
(12) Plant Patent Application Publication
(10) Pub. No.: US 2020/0236830 P1
(43) Pub. Date: Jul. 23, 2020

(54) Cannabis plant named 'MR2018002'

(50) ;

(71) Applicant: MedReleaf Corp., Toronto (CA)

(72) Inventors: Jeremy N. Friedberg, Guelph (CA); Andrew Hand, Bradford (CA); Alan Graziano, Toronto (CA)

(73) Assignee: MedReleaf Corp., Toronto (CA)

(21) Appl. No.: 16/873,050

(22) Filed: Jan. 21, 2020

(30) Foreign Application Priority Data
Jan. 18, 2019 (CA) 19-9708

(51) Int. Cl. A01H 6/28

(52) U.S. Cl. PLT/258

(57) ABSTRACT

A new cultivar of Cannabis named ‘MR2018002’ that is characterized by about 6% THC and 13% CBD by dry weight and resistance to microbial growth.

Genus and species: Cannabis sativa.

Variety denomination: ‘MR2018002’.

### CROSS-REFERENCE TO RELATED APPLICATIONS

This application claims the benefit of priority under 35 US.S.C 199(f) to Canadian Plant Breeders' Rights Application Number 19-9708, which was filed for the instant plant variety on Jan. 18, 2019.

### BACKGROUND OF THE INVENTION

The present invention relates to a new and distinct Cannabis (Cannabis sativa) cultivar designated as ‘MR2018002’.

‘MR2018002’ is a selection resulting from a controlled-cross between a male Cannabis sativa variety of unknown background and a female Cannabis sativa variety of unknown background.

Seeds from the cross were sown in Markham, Ontario, Canada and plants were screened for a number of traits including THC and CBD levels, yield, flowering time, disease resistance and flower morphology. An individual plant having about 6% THC and 13% CBD by dry weight and resistance to microbial growth was chosen to be ‘MR2018002’.

In September 2016, ‘MR2018002’ was first asexually propagated by apical stem cuttings approximately 10 cm long and having multiple auxiliary meristems, in Markham, Ontario, Canada. ‘MR2018002’ is stable and reproduces true to type in successive generations of asexual reproduction.

### BRIEF SUMMARY OF THE INVENTION

This invention relates to a new and distinctive Cannabis cultivar designated as ‘MR2018002’.

‘MR2018002’ exhibits approximately about 6% THC and 13% CBD by dry weight. ‘MR2018002’ also exhibits microbial resistance.

### BRIEF DESCRIPTION OF THE DRAWINGS

The accompanying color photographs depict characteristics of ‘MR2018002’ as nearly true as possible to make color reproductions. The photographs are of plants aged 53 days post-floral transition and grown in flower rooms under standard cultivation methods in Bradford, Ontario Canada.

FIG. 1 shows multiple, whole plant clones of ‘MR2018002’.

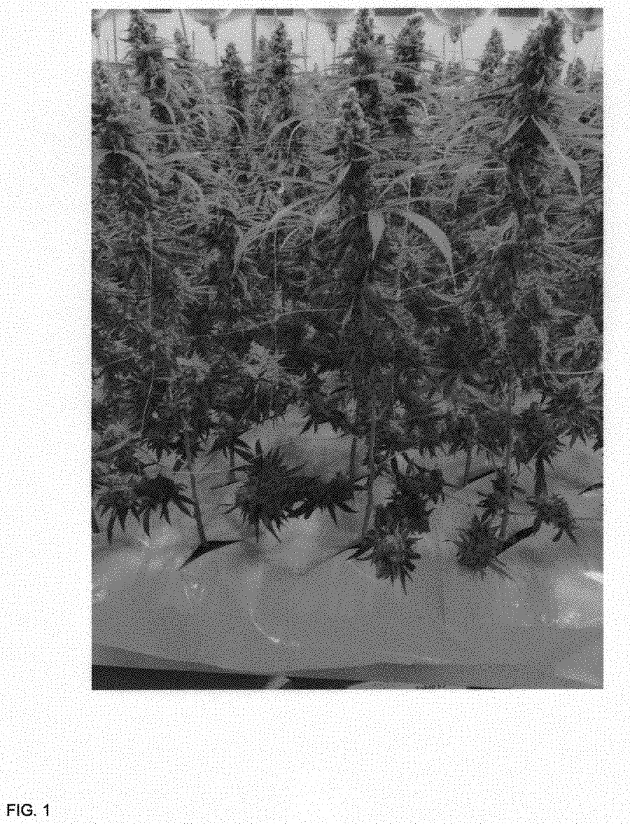

FIG. 2 shows a close-up view of an inflorescence and foliage of ‘MR2018002’.

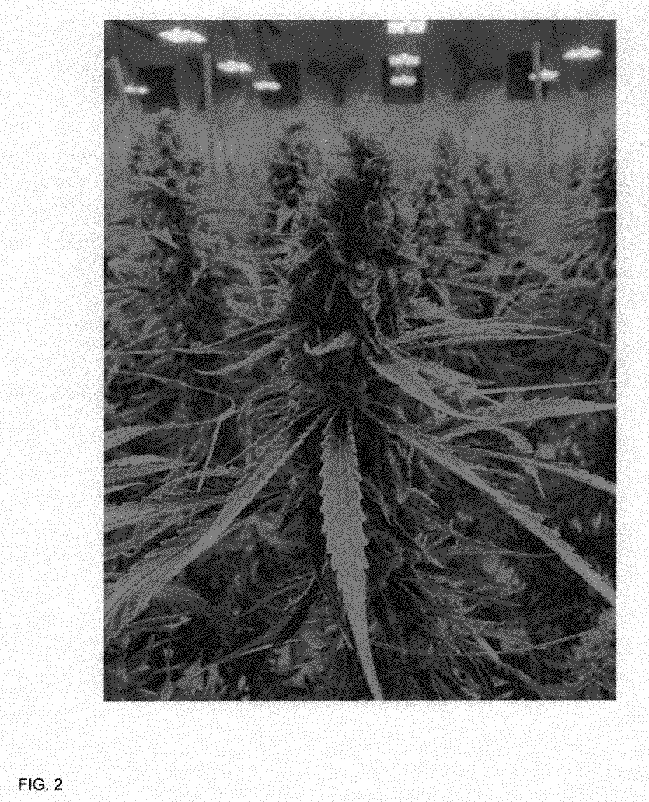

### DETAILED BOTANICAL DESCRIPTION

The following is a detailed description of the new cultivar ‘MR2018002’.

‘MR2018002’ has not been tested under all possible environmental conditions. Phenotypic differences may be observed with variations in environment without any variance in genotype.

The traits of ‘MR2018002’ have been repeatedly observed and represent the distinguishing characteristics of ‘MR2018002’.

The data that follows was collected in Markham, Ontario.

Plants were flowered under standard indoor environmental conditions with High Pressure Sodium lamps. Clones were cut from healthy mothers and allowed to roots for 14 to 18 days prior to being introduced to the flower room. The room was maintained at 18 hour days/6 hour nights for a week to allow the cuttings to establish adequate vegetative tissue. The floral transition was initiated by switching to 12 hour days and 12 hour nights. Flowering lasted 8-9 weeks before harvest, at which point samples were taken for chemical analysis, and yield was quantified. Morphological measurements were taken from plants aged 7 weeks post-floral transition.

Standard in-house developed nutrients were used throughout growth, and the plants were defoliated and pruned as necessary throughout the cycle.

In the following description, the color determination is in accordance with the 2017 Munsell Plant Tissue Color Book, except where general color terms of ordinary dictionary significance are used.
Classification:
   Denomination.—‘MR2018002’.
   Family.—Cannabacaeae.
   Genus.—Cannabis.
   Species.—Cannabis sativa.
   Common name.—Marijuana.
Parentage: Male Cannabis sativa variety of unknown background and a female Cannabis sativa variety of unknown background.
Propagation: ‘MR2018002’ is asexually (clonally) propagated from vegetative cuttings. Roots are fine and well-branched.
Plant:
   Height.—65 cm-75 cm.
   Width.—Approx. 40 cm.
Stems:
   Length/height.—50 cm-60 cm.
   Width/diameter.—7 mm-9 mm.
   Color.—2.5 GY 6/6, 5 RP 3/6.
   Shape.—Tubular.
   Texture.—Fibrous and Pubescent.
   Lateral branch length.—25 cm-35 cm.
Foliage:
   Type/form.—Palmately Compound.
   Arrangement.—Alternate.
   Attachment.—Petiolate.
   Leaf width.—9 cm-14 cm.
   Leaf length.—6 cm-15 cm.
   Number of leaflets per leaf.—5-7.
   Leaflet shape.—Lanceolate.
   Leaflet length.—4 cm-14 cm.
   Leaflet width.—1 cm-3 cm.
   Leaflet margin.—Serrate.
   Leaflet apex.—Acuminate.
   Leaflet base.—Attenuate.
   Leaflet color, upper surface.—7.5 GY 3/4, 7.5 GY 4/6.
   Leaflet color, lower surface.—5 GY 5/4.
   Venation pattern and description, upper and lower leaflet surfaces.—Pinnate.
   Texture (both surfaces).—Pubescent, primarily on the abaxial side and along leaf vasculature.
   Stipules.—Present, 2 per node on either side of a petiole, attenuated.
Petiole:
   Length.—1 cm-4.5 cm.
   Diameter.—0.2 mm-0.29 mm.
   Texture.—Fine, short non-glandular trichomes.
   Color.—5 GY 6/6, 5 RP 3/6.
Inflorescence:
   Blooming habit.—Short day photo period sensitive.
   Attachment.—Subsessile.
   Bracts.—Covered with trichomes and resin glands with 2 to 3 stigmas.
   Inflorescence color.—5 GY 5/8, 5 GY 5/10.
   Number of inflorescences per plant.—18-35.
   Diameter.—3 cm-6 cm.
   Length.—4 cm-8 cm.
   Inflorescence anthocyanin.—Absent.
   Average flower width.—3.7 cm.
Reproductive organs:
   Pistils.—1.
   Quantity (of stigmas) per flower.—2-4.
   Stigma length.—0.4 cm-0.8 cm.
   Stigma width.—0.7 mm-0.9 mm.
   Stigma color.—2.5 GY 8/2, 7.5 YR 5/6.
THC and CBD content:
   Average % THC by dry weight.—6%.
   Average % CBD by dry weight.—13%.
Yield:
   Average flower yield.—642 g/m² (grams dried, trimmed flower per m² of growing space).
   Average trim yield.—238 g/m² (grams trichome rich leaf material per m² that is removed during harvest/trimming).
Disease and insect/pest resistance: ‘MR2018002’ exhibits resistance to aerobic bacteria, yeast, mold and coliform bacteria as shown in Table 1.

TABLE 1
‘MR2018002’ resistance to aerobic bacteria, yeast,
mold and coliform bacteria.

| | |
|---|---|
| Average Aerobic Bacteria | 4407.5 |
| Count (per plant, in colony | |
| forming units (CFUs)) | |
| Average Yeast and Mold Count | 2057.5 |
| (per plant, in CFUs) | |
| Average coliform bacteria | 10 |
| count (per plant, in CFUs) | |

### COMPARISON WITH KNOWN VARIETY

A close variety of Cannabis known to the inventors is the commercial variety ‘Girl Scout Cookies’. ‘MR2018002’ can be distinguished from ‘Girl Scout Cookies’ by its increased resistance to microbial growth, as shown in Table 2:

TABLE 2
Comparison between ‘MR2018002’ and ‘Girl Scout Cookies’:
Microbial resistance

| | | | Average | |
|---|---|---|---|---|
| | | Average | Yeast | Average |
| | # of | Aerobic | and | Coliform |
| | Cycles | Bacteria | Mold | Bacteria |
| Variety | Grown | Count* | Count** | Count* |
| MR2018002 | 9 | 4407.5 | 2057.5 | 10 |
| Girl Scout | 15 | 59270 | 52450 | 760 |
| Cookies | | | | |

**Aerobic Bacteria Count, Yeast and Mold Count and Coliform Bacteria Count were determined per plant, in colony forming units (CFUs).

Variety ‘MR2018002’ can also be distinguished from the variety ‘Girl Scout Cookies’ with respect to the morphological traits shown in Table 3.

TABLE 3
Comparison between ‘MR2018002’ and ‘Girl Scout Cookies’:
Morphological traits

| | | ‘Girl Scout |
|---|---|---|
| Characteristic | ‘MR2018002’ | Cookies’ |
| Overage | 67.9 cm | 69.6 cm |
| Average Height | | |
| Average Active | 58.1 cm | 56.5 cm |
| Height | | |
| Average # of | 34.4 | 22.3 |
| Nodes | | |
| Average | 4.6 cm | 6.6 cm |
| Internodal Space | | |
| Average Flower | 3.7 cm | 5.3 cm |
| Width | | |

In addition, the flowers produced by variety ‘MR2018002’ are denser and stickier than the flowers produced by ‘Girl Scout Cookies’.

1. A new and distinct cultivar of Cannabis plant named ‘MR2018002’ as described and illustrated herein.
